(19) United States
(12) Patent Application Publication
(10) Pub. No.: US 2023/0298763 A1
(43) Pub. Date: Sep. 21, 2023

(54) METHOD AND SYSTEM FOR CALCULATING THE FORCE EXCHANGED BETWEEN A FLUID AND A SURROUNDING CONTAINER, PARTICULARLY IN CARDIOVASCULAR IMAGING

(71) Applicant: AMID S.R.L., Sulmona (IT)

(72) Inventors: Gianni PEDRIZZETTI, Prato (IT); Giovanni TONTI, Sulmona (IT)

(21) Appl. No.: 17/478,994

(22) PCT Filed: Mar. 20, 2019
(86) PCT No.: PCT/EP2019/025073

§ 371 Date: Sep. 20, 2021

(51) Int. Cl. G16H 50/50; G16H 30/40; G06F 30/23

(52) U.S. Cl. CPC G16H 50/50; G06F 30/23; G16H 30/40

(57) ABSTRACT

The disclosure relates to a method for determining one or more parameters related to the forces exchanged between a fluid and a surrounding container from sequences of images of the boundary surface of such container, the method comprising: a) expressing the boundary surface S(t) of the container as a series of meshes s, each mesh identified by a position vector x(s,t); b) calculating, or receiving in input, the instantaneous velocity vector v(s,t) at each position x(s,t); c) calculating, or receiving in input, the vector n(s,t) normal to the surface at each position x(s,t); d) calculating at each position x(s,t) a surface parameter f(s,t) as a function of the velocity vector v(s,t), the position vector x(s,t) and the normal vector n(s,t); e) deriving the parameter or the parameters related to the forces exchanged between the fluid and the surrounding container from such surface parameter f(s,t). A corresponding system and computer program are also disclosed.

The disclosure relates to a method, a computer program and a system for determining the force exchanged between a fluid flowing inside a container, particularly blood in heart chambers.

A fluid moving inside a solid deformable container and possibly moving in and out of such a container exchanges forces with the surrounding boundary. Such forces may have an important impact of the resistance and long-term deformation of the container.

Particularly relevant is the case of blood flowing inside a vessel like a chamber of the heart. The blood moves in virtue of the ability of the chamber, for example the left ventricle (LV) or the right ventricle (RV), of creating the proper intraventricular pressure gradients (IVPGs) that drive blood motion during both ejection and filling.

The relevance of IVPGs in cardiology or, equivalently, of the total hemodynamic force (HDF) vector which is the IVPG field integrated over the entire cavity, was recognized by numerous studies. Clinical studies about base-apex IVPGs were based on catheterization in animal models (Courtois et al., 1988; Guerra et al., 2013) Results showed that the dynamic rhythm of base-apex IVPG is a clear marker of normal function that is altered in either systolic or diastolic dysfunctions and abolished during heart failure. Despite their apparent importance, IVPGs have been rarely utilized in past clinical cardiology due to the invasiveness of their acquisition requiring a catheter or dynamometric elements.

Advancements in cardiovascular imaging, echocardiography and MRI, allow the measurement of blood flow field inside the heart chambers and, from post-processing, the non-invasive calculation of the corresponding pressure field. By using the law of conservation of momentum we have

$$F \left(t\right) = ρ \frac{d}{d t} {\int }_{V \left(t\right)} v d V \qquad \text{(1)}$$

where F(t) is the hemodynamic force vector, ρ is the fluid density, v(x,t) is thefluid velocity vector field measured at time t at all points x inside the chamber volume V(t), and the integration is taken over the moving volume of fluid, thus the points are assumed as moving fluid particles. It is often convenient to describe the same calculation in terms a volume of fluid that is fixed in space, because measurements of fluid velocity are more commonly feasible at fixed points than over moving particles. Using the Reynolds transport theorem, the integral (1) can be rewritten as

$$F \left(t\right) = {\int }_{V \left(t\right)} ρ \frac{\partial v}{\partial t} d V + {\int }_{s \left(t\right)} ρ v \left(v ⋅ n\right) d S , \qquad \text{(2)}$$

where now integration over the fixed points x inside the volume V(t) assumed as instantaneously fixed, S(t) is the closed surface bounding such volume and n is the outward unit normal vector.

Given that blood is an incompressible medium, equation (2) is often rewritten as a single integral over the volume as

$$F \left(t\right) = ρ {\int }_{V \left(t\right)} \left(\frac{\partial v}{\partial t} + v ⋅ ∇ v\right) d V , \qquad \text{(3)}$$

because it allows to compute the hemodynamic force vector on the basis of the velocity vector field measured inside the volume. Based on formulas (2) or (3) the concept of hemodynamic forces (HDFs) was then introduced as a mathematically well-defined global measure which corresponds to the integrated IVPGs inside the LV and represents the force exchanged between the blood and the surrounding tissue (Cimino et al., 2012; Pedrizzetti et al., 2015).

Using formula (3) HDFs were computed non-invasively in echocardiography using methods able to evaluate the blood flow velocity like Echo-PIV (Pedrizzetti et al., 2016), Doppler-VFM (Mele et al. 2018), or color Doppler M-mode echocardiography (Firstenberg et al., 2000; Greenberg et al., 2001). More recently, three-dimensional/three-directional phase-contrast magnetic resonance imaging (MRI), commonly referred as 4D flow MRI, was used to evaluate HDFs in the LV on both normal and diseased subjects (Arvidsson et al., 2016; Eriksson et al., 2016). Based on these technologies, the assessment of HDFs is becoming an important marker of cardiovascular function.

However, these approaches present technical difficulties.

Fluid velocity evaluated by ultrasound is approximated when using Echo-PIV which also preferably requires the infusion of a contrast agent, whereas it is essentially mono-directional and with limited accuracy when using Doppler-based methods. The usage of 4D Flow MRI is more reliable and accurate; nevertheless, it requires costly equipments in a fixed installation, and requires time-consuming procedures for both acquisition and post-processing. It produces a large amount of information, a three-dimensional velocity vector measured at all points in three-dimensional space, and it may not be recommended for estimating a single HDF parameter. Recently, a simplified method was introduced to estimate HDFs from the motion of the surrounding boundaries that can be recorded with relative ease in echocardiography or are available from simple cine cardiac MRI (Pedrizzetti et al. 2017). However, that method is mathematically complex and approximate, and it applies only to the LV when it has a sufficiently regular geometry.

It is thus an object of the present disclosure to provide for methods and systems for determining one or more parameters related to the forces exchanged between a fluid and a surrounding container, particularly blood in the heart cavities, which are simple and easily applicable, particularly in the clinical practice.

The aim is reached with a computer implemented method comprising:
a) providing sequences of images of the boundary surface S(t) of the container;
b) expressing the boundary surface S(t) of the container as a series of meshes each identified by a position vector x(s,t). Such meshes may be geometrical figures like polygons, particularly triangles, circles, ellipses, with the position vector x(s,t) identifying such figures;
c) calculating, or receiving in input, the instantaneous velocity vector v(s,t) at each position x(s,t);
d) calculating, or receiving in input, the vector n(s,t) normal to the surface at each position x(s,t);
e) calculating at each position x(s,t) a surface parameter f(s,t) as a function of the velocity vector v(s,t), the position vector x(s,t) and the normal vector n(s,t), particularly as
$$x \left(\frac{\partial v}{\partial t} ⋅ n\right) + v \left(v ⋅ n\right) ;$$
f) deriving the parameter or the parameters related to the forces exchanged between the fluid and the surrounding container from such surface parameter f(s,t), particularly by integrating the surface parameter f(s,t) over the surface boundary S(t) and/or determining the projection of the surface parameter f(s,t) on the normal n(s,t) to the surface at each position x(s,t).

If the container is the heart, particularly a chamber of the heart, the fluid the blood and the forces exchanged between the fluid and the container the hemodynamic forces inside the heart.

By considering only information related to the boundary surface, it is thus possible to estimate intraventricular pressure gradients (IVPGs) or, equivalently, the total hemodynamic force vector (HDF), or a parameter related thereto, that drive blood motion during both ejection and filling.

This has something surprising as the fluid-dynamics provides well-known formulas and calculations for obtaining such information only if spatial fluid velocity inside the heart is known.

A parameter related to a local force vector f(x,t) may be advantageously calculated as

$$ρ \left[x \left(\frac{\partial v}{\partial t} ⋅ n\right) + v \left(v ⋅ n\right)\right] ⋅ n$$

where ρ is the density of the fluid. From such parameter a global force vector F(t) can be derived as

$$F \left(t\right) = ρ {\int }_{s \left(t\right)} \left[x \left(\frac{\partial v}{\partial t} ⋅ n\right) + v \left(v ⋅ n\right)\right] d S$$

Alternatively, the normal component of the local force vector, related in the integral sense to pressure distribution p(x,t), may be calculated as

$$ρ \left[x \left(\frac{\partial v}{\partial t} ⋅ n\right) + v \left(v ⋅ n\right)\right] ⋅ n$$

As a further option, the tangential component or the norm of the local force vector f(x,t) may be considered as the output parameter.

According to an improvement, the parameter or the parameters may be normalized over the volume of the container V(t) calculated, for example, as

$$V = \frac{1}{3} {\int }_{s} x ⋅ n d S$$

In case of containers like the heart having apertures, indicating with S₁ the surface of the solid part and with S₂ the surface of the at least one aperture, the instantaneous velocity of the fluid crossing the open boundary surface S₂ can be received in input or calculated using the principle of conservation of mass in the form of an average normal velocity across the aperture

$${\int }_{{S}_{1}} v ⋅ n d S$$

as

$$- {\int }_{{S}_{2}} v ⋅ n d S$$

In case of the heart, the open boundary surfaces are the valves that are advantageously segmented as single circular or polygon mesh.

According to an embodiment, the sequences of images of the boundary surface of the container are obtained by operating a three-dimensional reconstruction of the container boundary surface based on bi-dimensional or three-dimensional image datasets.

An embodiment is related to a computer product directly loadable in the memory of a digital computer and comprising software code portions for performing the method according to according to embodiments herein when the product is run on a computer.

In accordance with a further embodiment, a system for determining one or more parameters related to the forces exchanged between a fluid and a surrounding container is also provided. The system includes a graphical user interface (GUI) configured to receive user inputs, a memory to store program instructions, an input for receiving one or more sequences of two-dimensional or three-dimensional images of the container and an output for outputting force-related parameters in numeric and/or graphical format. A processor is configured to execute the program instructions to perform the steps of the method according to embodiments herein.

Further improvements of the invention will form the subject of the dependent claims.

The characteristics of the invention and the advantages derived therefrom will be more apparent from the following description of non-limiting embodiments, illustrated in the annexed drawings, in which:

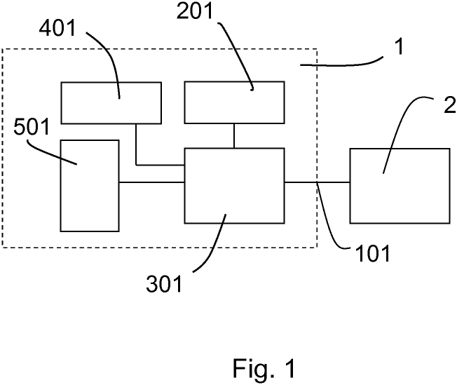

FIG. 1 illustrate exemplified block diagram of a first embodiment of the device.

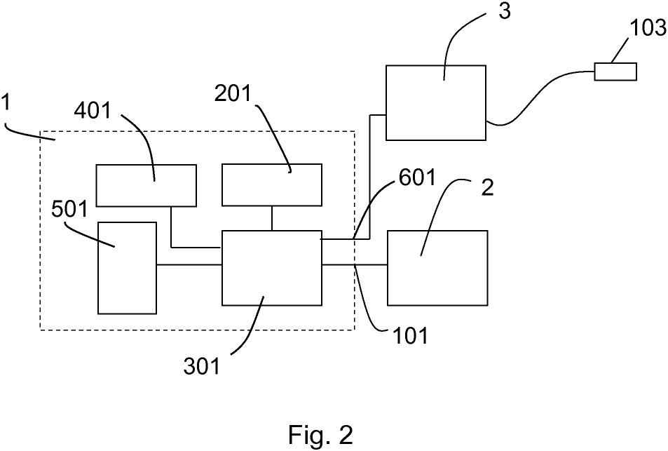

FIG. 2 illustrates an exemplified block diagram of a second embodiment of the device.

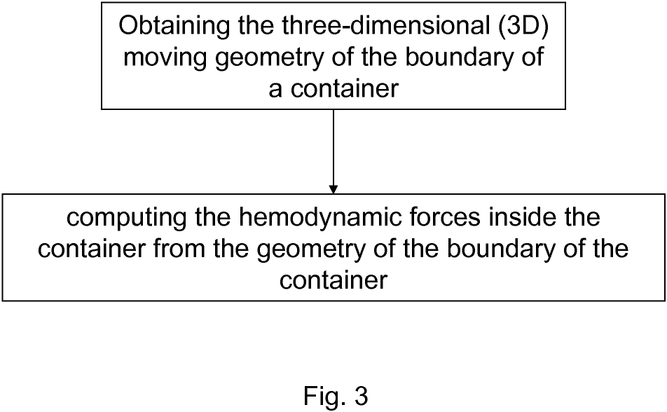

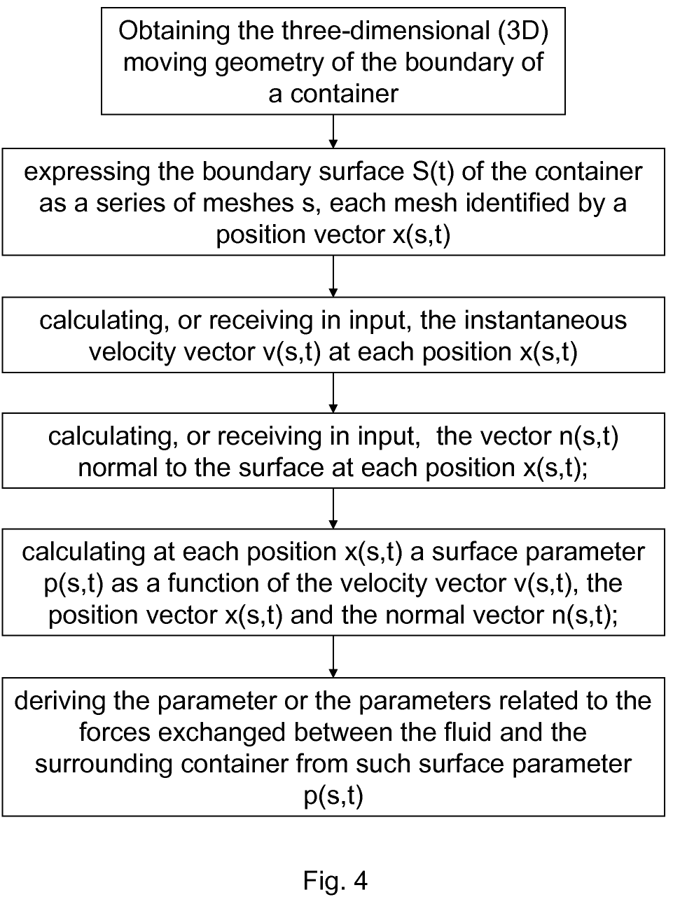

FIGS. 3-4 illustrates flowcharts of the operations of methods according to embodiments herein.

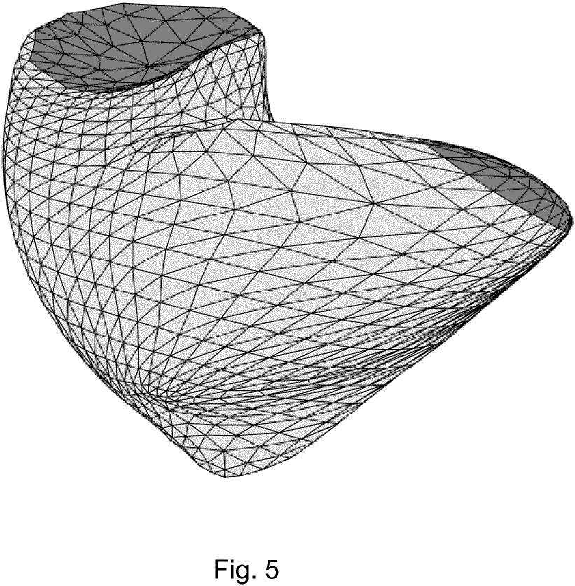

FIG. 5 shows one example for the surface of a right ventricle (RV) made of triangular elements.

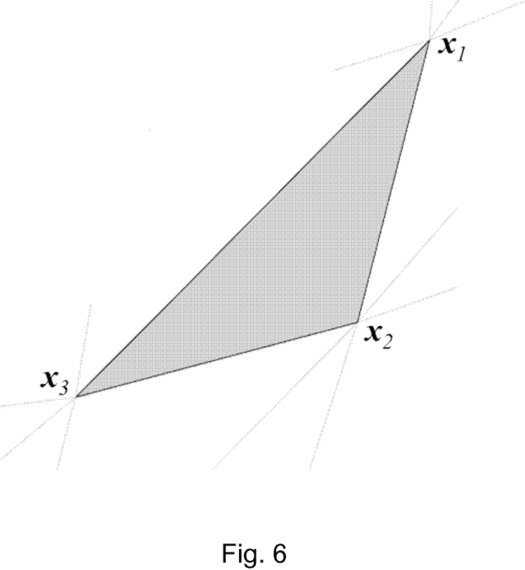

FIG. 6 shows the surface of a RV of a child extracted with semi-automated segmentation shown at end-systole (smaller darker RV) and end-diastole (wider semi-transparent surface).

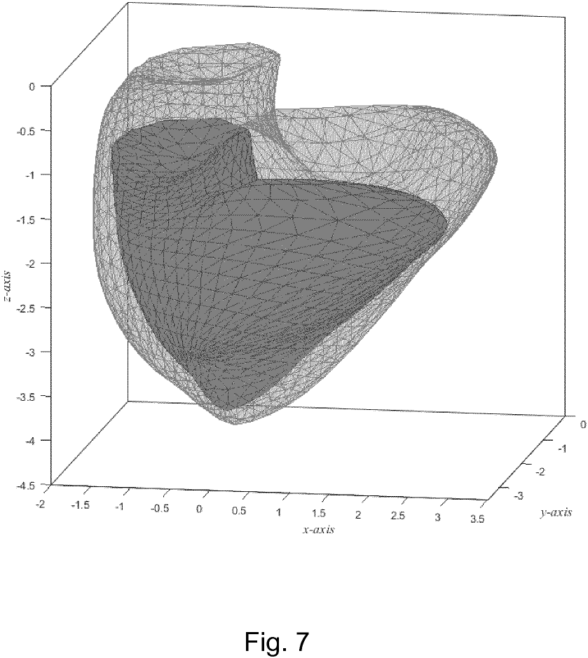

FIG. 7 shows meshes in the form of triangular elements described by the 3D position vectors of their vertices.

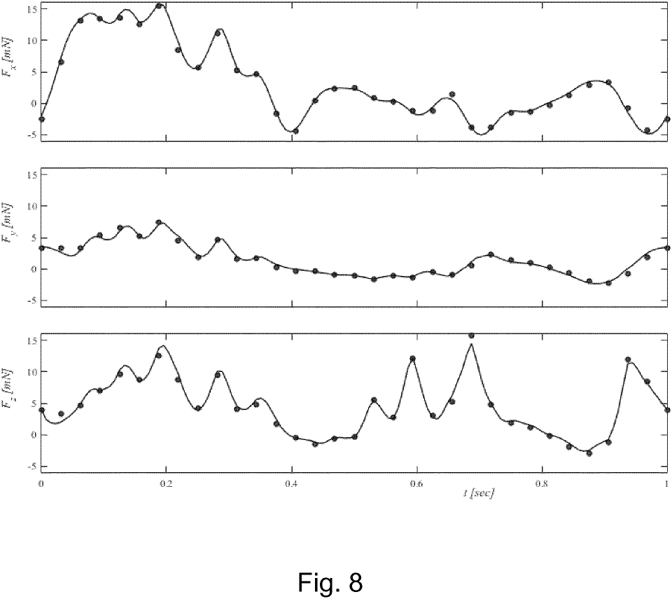

FIG. 8 shows the hemodynamic force computed during one heartbeat inside the RV. Comparison between results from computational fluid dynamics (continuous line) and those obtained by the approach disclosed here (black dots). The three components of the force are reported separately.

The invention will be mainly described with reference to the application to measure hemodynamic parameters and thus with the heart as target of the analysis. This is, however, not to be considered limiting of the scope of protection as the same considerations that will be discussed in details hereinafter can be easily extended to any fluid in any deformable container with or without inlet/outlet apertures.

With reference to FIG. 1, the system according to an embodiment comprises a graphical user interface (GUI) 401 to receive user inputs, memory 201 to store program instructions, a processing unit 301 that reads input data 101 and process them to estimate hemodynamic forces parameters in the heart of a patient by executing the program instructions to perform one or more steps of the method according to embodiments herein. An output, for example in form of a monitor 501, can show the results of the analysis in graphics and/or numeric form. The processing unit 301 may be a dedicated microprocessor system or, more generally, a PC also of the general purpose type. The characteristics of the unit 301 will obviously reflect on the processing speed.

The input data may be already in the form of sequences of images of the boundary surface of the heart or two-dimensional or three-dimensional images of the full heart. In this case the processing unit 301 is configured to preliminarily make a three-dimensional reconstruction of the heart boundary surface, for example, from imaging datasets received from an imaging apparatus indicated in the figures with reference 2. Examples are ultrasound, MRI, Computed Tomography (CT), optical or laser-based apparatus as known in the art. In an embodiment, the system may also be integrated in one of such apparatus to obtain a very compact configuration.

As exemplary shown in FIG. 2, the system may also comprise a further input 601 for receiving the values of the velocity of the fluid at apertures crossing the boundary surface of the container, with the processing unit 301 advantageously configured to use such values as the velocity of the meshes covering such apertures.

An echographic apparatus having Doppler capabilities or a phase-contrast MRI apparatus 3 for acquiring velocity values of the fluid crossing apertures of the container to be transferred to the second input 601 of the system may be advantageously provided in combination. In an embodiment, this apparatus is the same as the one providing the imaging datasets at input 101 with evident benefits in term of compactness and cost reduction.

Depending on the type of data available at the input, the processing unit 301 is configured to elaborate data information of the heart of a subject to estimate hemodynamic forces parameters by performing the operations as shown in the flowcharts of FIGS. 3 to 4.

With reference to the flowchart of FIG. 3, an embodiment comprises two basic operations:
(A) obtaining non-invasively the three-dimensional (3D) moving geometry of the boundary of the container;
(B) computing the hemodynamic forces from such geometry based on an original mathematical solution.

### (A) OBTAINING NON-INVASIVELY THE THREE-DIMENSIONAL (3D) MOVING GEOMETRY OF THE BOUNDARY OF THE CONTAINER

The internal geometry of a cardiovascular district, like LV or RV, can be visualized by existing imaging technologies, like regular ultrasound or MRI or Computed Tomography (CT). Other technologies, like optical or laser-based, can be used in different environments when appropriate. The moving geometry can then be identified by manual delineation or by application of automated segmentation methods, many of them existing based on edge detection, pattern recognition, neural network, atlas matching, active deformation or other techniques ending with the recognition of a boundary, border, edge. The movement is given by applying segmentation method in different instants, it can also be estimated by application of optical flow methods capable of tracking the motion of boundaries segmented in one or some instants of time as disclosed, for example, in Seo et. al., 2014 and Muraro et. al, 2016, to be considered incorporated herein by reference.

The 3D geometry can be obtained by application of these methods for estimating the boundaries directly to 3D images, or datasets, when a 3D imaging technology is used. See for examples applications available in literature (Satriano et al. 2017; Pedrizzetti et al. 2014; Zheng 2018; Satriano et al. 2019) to be considered incorporated herein by reference. The 3D geometry can also be reconstructed by the boundaries estimated from one or several 2D images, which are then appropriately recombined in 3D space. Like the 3 longitudinal slices commonly used for imaging in the LV, or multiple short axis slices. See for example the three dimensional reconstruction from simple cine cardiac MRI (Pedrizzetti et al. 2017; Biffi 2019) to be considered incorporated herein by reference. There are also numerous solutions available in the market to this purpose, of which some examples are reported here (“4D LV-Analysis” and “4D RV-Function”, TOMTEC Imaging Systems GmbH, Germany; “Medis Suite MR”, Medis medical imaging systems bv, Leiden, The Netherlands; “Segment CMR” and “Segment CT”, Medviso AB, Lund, Sweden), to be considered incorporated herein by reference.

The result of this step will to be a 3D reconstruction of the container boundary surface S(t), which will be composed by a solid boundary, S₁(t), like the endocardium of the LV, and an open boundary, S₂(t), from which fluid can enter or exit the contained, like the valve of the LV. The surface will be described by a series of position vectors x(s,t) over the surface. These position vectors change their coordinate value during time, t, accordingly to the motion of the surface. Such positions are marked by an identifier, or an index, generally indicated by s; this identification permits to draw a connection structure to description of the surface in terms of triangular, rectangular or other type of surface elements, each element identified by the corresponding position vectors of its vertices.

FIG. 5 shows one example for the surface of a RV made of triangular elements. FIG. 6 shows that each individual triangular elements is described by the 3D position vectors of its vertices.

### (B) COMPUTING THE HEMODYNAMIC FORCES HDF FROM THE GEOMETRY OF THE BOUNDARY OF THE CONTAINER

First, the instantaneous velocity of each single position is computed by time differentiation of the boundary position

$$v \left(s , t\right) = \frac{\partial x}{\partial t} , \qquad \text{(4)}$$

such time derivative can be performed numerically based on the position known on a series of instant of time using a formula for numerical derivatives. In the case of a boundary describing a solid non-permeable boundary, the velocity (4) corresponds to the velocity of the viscous fluid. For the open parts of theboundary, the velocity has to be obtained by direct measurement, from other information or additional considerations.

Formulas (1), (2) or (3), contain integrals of volume that require the knowledge of values internal to the fluid domain and cannot be evaluated by the knowledge of values on the surface only. However, a careful inspection shows that the first term in (2) can be rewritten in terms of surface integral as follows.

$${\int }_{V \left(t\right)} \frac{\partial v}{\partial t} d V = {\int }_{S \left(t\right)} x \left(\frac{\partial v}{\partial t} ⋅ n\right) d S , \qquad \text{(5)}$$

This is achieved by an original application of the Gauss theorem, also known as divergence theorem, that takes advantage that the fluid is incompressible. This can be verified for a generic component i, given that velocity is divergence-free and considering that the integral is over fixed points in space, we can write

$${\int }_{V \left(t\right)} \frac{\partial {v}_{i}}{\partial t} d V = {\int }_{V} \frac{\partial }{\partial {x}_{k}} \left({x}_{i} \frac{\partial {v}_{k}}{\partial t}\right) d V = {\int }_{S} {x}_{i} \frac{\partial {v}_{k}}{\partial t} {n}_{k} d S , \qquad \text{(6)}$$

where the last equality uses the Gauss theorem and summation over repeated index (here k) is implicitly assumed (Einstein notation).

Thus combination of formula (5) into (2) provides a mean to compute the hemodynamic force from surface integrals thus from the knowledge of values at the boundaries only. The complete result can be written in a single formula

$$F \left(t\right) = ρ {\int }_{S \left(t\right)} \left[x \left(\frac{\partial v}{\partial t} ⋅ n\right) + v \left(v ⋅ n\right)\right] d S . \qquad \text{(7)}$$

It is immediate to verify that analogous results can be obtained by different combination of the same procedure to formula (1) or (3) with minor formal differences and equivalent results, posing special attention to the difference between a volume described by fixed positions in (2) and (3) and one composed of moving fluid particles in (1).

The hemodynamic force is often normalized with the volume V(t) of the chamber under analysis which can be computed by numerous means as well as, for example, by application of Gauss theorem

$$V = \frac{1}{3} {\int }_{S} x ⋅ n d S . \qquad \text{(8)}$$

The procedure, formally synthesized in formula (7), is an exact result never disclosed before that permits to compute HDF vector from the knowledge of the moving boundary as obtained in the previous step (A). The surface integrals present therein can be evaluated by numerous numerical methods. A simple mean is that of transforming the integral in a summation over all individual surface elements, like the triangles in FIG. 5, once all properties inside the integral are estimated for each surface elements from the values at its vertices. Any quadrature formulas or numerical integration methods can also be employed depending on convenience.

The surface integrals, like those in (2), (5), (7), (8), comprise integration over the closed, solid part of the container boundary, S₁, and over the open part across which the fluid can flow, S₂. Whereas the fluid velocity over the solid part corresponds to the boundary velocity and is commonly obtained directly from images, possibly after differentiation (4), the fluid velocity over the open parts may not be directly available. Sometime it can be directly provided by imaging techniques, like in some application of Doppler ultrasound or by phase-contrast MRI that is frequently used to measure the out-of-plane velocity component. When these measurements are not available one can use mass conservation

$${\int }_{{S}_{1}} v ⋅ n d S = - {\int }_{{S}_{2}} v ⋅ n d S , \qquad \text{(8)}$$

to estimate the average normal velocity across the open part. Moreover the fluid velocity can be assumed as approximately uniform across the orifice and mostly unidirectional such that the velocity is given by v = (v.n)n. The same argument applies to a single open orifice or to multiple open orifices.

In general, the existence of a formula like (7) permits to estimate a distribution of forces, relative to a time varying constant value, over the bounding surface. Recall that the total force can be computed by definition, and ignoring the viscous force that are largely negligible in the cardiac chamber (Domenichini e Pedrizzetti 2015), as the integral of pressure, p(x,t), over the surface

$$F \left(t\right) = {\int }_{S \left(t\right)} p n d S . \qquad \text{(9)}$$

The formal similarity between (7) and (9) suggests the presence of a distribution of local force term

$$f \left(x , t\right) = ρ \left[x \left(\frac{\partial v}{\partial t} ⋅ n\right) + v \left(v ⋅ n\right)\right] \qquad \text{(10)}$$

which plays a role analogous to pressure with (7) and (9) stating that analogy is valid at the integral level and not punctual. The distribution of pressure(10) can represent an indicator for revealing differences in the regional function of the cardiac chamber. Other indicators based on (10), like the normal component of the local force vector, stressing an analogy with pressure based on (9), the tangential component, stressing an analogy with the wall shear stress, or the magnitude, expresses as a norm, of the local force vector

$$f \left(x , t\right) = ρ \left‖x \left(\frac{\partial v}{\partial t} ⋅ n\right) + v \left(v ⋅ n\right)\right‖ \qquad \text{(11)}$$

or other combinations, can also be used as indicators for specific classes of pathologies or as indicator of cardiac remodelling.

The complete procedure (A)+(B) has been verified by calculating the hemodynamic forces in a complex RV geometry during the heartbeat and comparing the results obtained by the approach disclosed here with respect to that obtained by applying equation (3) to a complete three-dimensional three-directional velocity vector field calculated by computational fluid dynamics (CFD).

The RV was recorded by echocardiographic imaging and the segmentation was performed by a semi-automated algorithm of the type described in (Seo et al 2014; Muraro et al 2016). FIG. 7 shows the geometry of the RV segmented surface at two instants. The same geometries were used to perform the CFD study with high resolution in order to obtain the velocity vector field inside the moving RV volume at all instants during one heartbeat. To this aim, CFD resolves the equations governing the fluid dynamics for an incompressible Newtonian fluid inside the geometry that is immersed in the domain, all details of the CFD technique are reported in (Mangual et al. 2012). Based on the CFD results (that takes about 40 hours in a workstation) the hemodynamic force is computed by volume integration (3), this step requires care in the segmentation of the internal volume based on the known surface. The same results are obtained by application of formula (7) directly from the previously segmented surface (less than 1 second in the same workstation). The comparison is reported in FIG. 8, as expected results are very comparable, minor differences are due to the different time resolution used when computing the time derivatives, differences in the spatial and volumetric numerical integrations, and to variability in the segmentation of the internal volume with a fixed grid in the CFD.

The forces exchanged between a fluid flowing inside a container are an important measure of the interaction between fluid and surrounding structures. In particular in the cardiovascular system, the hemodynamic forces, or equivalently pressure gradients, are considered of fundamental relevance for describing the function of cardiovascular districts.

Methods for computing them are approximate or present a significant complexity (4D Flow MRI). Having a mean for computing them accurately in a simple and rapid approach would be beneficial in numerous aspects. In particular, a mean for estimating them from regular, routinely-used, cardiovascular imaging would allow its application in medicine.

### REFERENCES

Courtois, M., Kovacs, S.J., Ludbrook, P.A., 1988. Transmitral pressure-flow velocity relation. Importance of regional pressure gradients in the left ventricle during diastole. Circulation 78, 661-71. doi:10.1161/01.cir.78.3.661.

Guerra, M., Bras-Silva, C., Amorim, M.J., Moura, C., Bastos, P., Leite-Moreira, A.F., 2013. Intraventricular pressure gradients in heart failure. Physiol. Res. 62, 479-487.

Cimino S, Pedrizzetti G, Tonti G, Canali E, Petronilli V, De Luca L, Iacoboni C, Agati L. In vivo Analysis of Intraventricular Fluid Dynamics in Healthy Hearts. Eur J Mech B/Fluids 2012; 35:40-46.

Pedrizzetti G, Martiniello AR, Bianchi V, D′Onofrio A, Caso P, Tonti G. Cardiac Fluid Dynamics Anticipates Heart Adaptation. J Biomech 2015; 48:388-391.

Mele D, Smarrazzo V, Pedrizzetti G, Capasso F, Pepe M, Severino S, Luisi GA, Maglione M, Ferrari R. Intracardiac Flow Analysis: Techniques and Potential Clinical Applications. J Am Soc Echocardiogr 2018. DOI:10.1016/j.echo.2018.10.018

Firstenberg, M.S., Vandervoort, P.M., Greenberg, N.L., Smedira, N.G., McCarthy, P.M., Garcia, M.J., Thomas, J.D., 2000. Noninvasive estimation of transmitral pressure drop across the normal mitral valve in humans: Importance of convective and inertial forces during left ventricular filling. J. Am. Coll. Cardiol. 36, 1942-1949. doi:10.1016/S0735-1097(00)00963-3

Greenberg, N.L., Vandervoort, P.M., Firstenberg, M.S., Garcia, M.J., Thomas, J.D., 2001. Estimation of diastolic intraventricular pressure gradients by Doppler M-mode echocardiography. Am. J. Physiol. Heart Circ. Physiol. 280, H2507-15.

Arvidsson PM, Töger J, Carlsson M, Steding-Ehrenborg K, Pedrizzetti G, Heiberg E, Arheden H. Left and right ventricular hemodynamic forces in healthy volunteers and elite athletes assessed with 4D flow magnetic resonance imaging. AJP-Heart and Circ 2016 . doi:10.1152/ajpheart.00583.2016.

Eriksson, J., Bolger, A.F., Ebbers, T., Carlhäll, C.-J., 2016. Assessment of left ventricular hemodynamic forces in healthy subjects and patients with dilated cardiomyopathy using 4D flow MRI. Physiol. Rep. 4, 741-747. doi:10.14814/phy2.12685

Pedrizzetti G, Arvidsson PM, TögerJ, Borgquist R, Domenichini F, Arheden H, Heiberg E. On estimating intraventricular hemodynamic forces from endocardial dynamics: a comparative study with 4D flow MRI. J Biomech 2017; 60:203-210. DOI:10.1016/j.jbiomech.2017.06.046

Domenichini F, Pedrizzetti G. Hemodynamic Forces in a Model Left Ventricle. Phys Rev Fluids 2016 1 083201.

Seo Y, Ishizu T, Aonuma K. Current status of 3-dimensional speckle tracking echocardiography: a review from our experiences. J Cardiovasc Ultrasound. 2014;22(2):49-57.

Muraru et al. 2016 New speckle-tracking algorithm for right ventricular volume analysis from threedimensional echocardiographic data sets: validation with cardiac magnetic resonance and comparison with the previous analysis tool. European Heart Journal - Cardiovascular Imaging 17, 1279-1289

Mangual J, Domenichini F, Pedrizzetti G. Describing the highly 3D flow in the right ventricle. ABME 2012; 40:1790-1801.

Stroud A (1971) Approximate Calculation of Multiple Integrals. Prentice-Hall. New Jersey, USA.

Chien D (1995) Numerical Evaluation of Surface Integrals in Three Dimensions. Math. Comput., 64(210):727-743.

Reeger JA, Fornberg B, Watts ML (2016) Numerical quadrature over smooth, closed surfaces. Proceedings Royal Society A 472, 20160401 (DOI: 10.1098/rspa.2016.0401).

Satriano A, Heydari B, Narous M, Exner DV, Mikami Y, Attwood MM, et al. Clinical feasibility and validation of 3D principal strain analysis from cine MRI: comparison to 2D strain by MRI and 3D speckle tracking echocardiography. Int J Cardiovasc Imaging 2017;33:1979-92.

Pedrizzetti G, Sengupta S, Caracciolo G, Park CS, Amaki M, Goliasch G, et al. Three-dimensional principal strain analysis for characterizing subclinical changes in left ventricular function. J Am Soc Echocardiogr 2014;27: 1041-1050..

Satriano A, Pournazari P, Hirani M, Helmersen D, Thakrar M, Weatherald J, White JA, Fine NM. Characterization of Right Ventricular Deformation in Pulmonary Arterial Hypertension Using Three-Dimensional Principal Strain Analysis. J Am Soc Echocardiogr 2019;32:385-393.

Zheng Q, Delingette H, Duchateau N, Ayache N. 3D Consistent & Robust Segmentation of Cardiac Images by Deep Learning with Spatial Propagation. arXiv:1804.09400v1 [cs.CV], 2018

Biffi C, Cerrolaza JJ, Tarroni G, de Marvao A, Cook SA, O’Regan DP, Rueckert D. 3D High-Resolution Cardiac Segmentation Reconstruction from 2D Views using Conditional Variational Autoencoders. arXiv:1902.11000 [cs.CV], 2019

1. A computer-implemented method for estimating hemodynamic forces between blood and a surrounding heart chamber from sequences of images of a boundary surface of such a heart chamber, the method comprising:
a) expressing the boundary surface S(t) of the heart chamber as a series of meshes s, each mesh identified by a position vector x(s,t);
b) calculating, or receiving in input, an instantaneous velocity vector v(s,t) at each position x(s,t);
c) calculating, or receiving in input, a normal vector n(s,t) normal to the boundary surface at each position x(s,t);
d) calculating at each position x(s,t) a surface parameter f(s,t) as a function of the velocity vector v(s,t), the position vector x(s,t) and the normal vector n(s,t); and
e) deriving a force vector as an estimate of the hemodynamic forces from the surface parameter f(s,t).

2. The method according to claim 1, wherein step e) comprises integrating the surface parameter f(s,t) over the surface boundary S(t).

3. The method according to claim 1, wherein step e) comprises determining a projection of the surface parameter f(s,t) on the normal n(s,t) to the surface at each position x(s,t).

4. The method according to claim 1, wherein step d) comprises calculating the surface parameter f(s,t) as
$$x \left(\frac{\partial v}{\partial t} ⋅ n\right) + v \left(v ⋅ n\right)$$
.

5. The method according to claim 1, wherein step e) comprises deriving a local force vector f(x,t) as an estimate of the hemodynamic forces as
$$f \left(x , t\right) = ρ \left[x \left(\frac{\partial v}{\partial t} ⋅ n\right) + v \left(v ⋅ n\right)\right] ⋅ n$$
where ρ is the density of the blood.

6. The method according to claim 1, wherein step e) comprises calculating a force vector F(t) as
$$F \left(t\right) = ρ {\int }_{S \left(t\right)} \left[x \left(\frac{\partial v}{\partial t} ⋅ n\right) + v \left(v ⋅ n\right)\right] d S$$
where ρ is the density of the blood.

7. The method according to claim 1, wherein step e) comprises calculating as parameter the normal component of the local force vector, related in the integral sense to pressure distribution p(x,t), as
$$ρ \left[x \left(\frac{\partial v}{\partial t} ⋅ n\right) + v \left(v ⋅ n\right)\right] ⋅ n$$
where ρ is the density of the blood.

8. The method according to claim 1, wherein step e) comprises calculating as parameter the tangential component or the norm of the local force vector f(x,t).

9. The method according to claim 1, wherein step e) comprises normalizing the estimated hemodynamic forces over the volume of theheart chamber V(t).

10. The method according to claim 9, wherein the volume of the heart chamber V(t) is calculated as
$$V = \frac{1}{3} {\int }_{S} x ⋅ n d S$$
.

11. The method according to claim 1, wherein step a) comprises expressing the boundary surface S(t) of the heart chamber as a series of geometrical figures, with the position vector x(s,t) identifying a center of such figures.

12. The method according to claim 1, wherein the heart chamber has a solid part having surface S₁ and at least one aperture having an open boundary surface S₂, step b) comprising receiving in input a velocity of the blood crossing the open boundary surface S₂ or calculating as the velocity vector v an average normal velocity across the aperture ∫_{S₁}ν · ndS as
$$- {\int }_{{S}_{2}} v ⋅ n d S$$
.

13. The method according to claim 1, wherein the sequences of images of the boundary surface of the heart chamber are obtained by operating a three-dimensional reconstruction of the heart chamber boundary surface based on bidimensional or three-dimensional image datasets.

14. (canceled)

15. The method according to claim 1, wherein those parts of the boundary surface corresponding to at least one heart valve are segmented as single circular or polygon mesh.

16. A computer product directly loadable in a memory of a digital computer and comprising software code portions for performing the method according to claim 1 when the product is run on the digital computer.

17. A system for estimating hemodynamic forces between blood and a surrounding heart chamber, comprising:
a) a first input for receiving one or more sequences of two-dimensional or three-dimensional images of the heart chamer;
b) memory to store program instructions;
c) a processing unit;
d) a graphical user interface configured to receive user inputs;
e) an output for outputting force-related parameters in numeric and/or graphical format, characterized in that the processing unit is configured to execute the program instructions to:
a) make a three-dimensional reconstruction of the heart chamber boundary surface S(t);
b) divide the boundary surface S(t) of the heart chamber in a series of meshess;
c) associate to each mesh a position vector x(s,t);
d) calculate an instantaneous velocity vector v(s,t) at each position x(s,t);
e) calculate a normal vector n(s,t) normal to the boundary surface at each position x(s,t);
f) calculate at each position x(s,t) a surface parameter f(s,t) as a function of the velocity vector v(s,t), the position vector x(s,t) and the normal vector n(s,t);
g) derive a force vector as an estimate of the hemodynamic forces from the surface parameter f(s,t);
h) output values based on such parameter or parameters.

18. The system according to claim 17, characterized in that it is provided in combination with an echographic, a CT or an MRI apparatus for acquiring sequences of two-dimensional or three-dimensional images of the heart chamber to be transferred to the first input of the device.

19. The system according to claim 17, further comprising a second input for receiving values of velocity of the blood at apertures crossing the boundary surface of the heart chamber, the processing unit being configured to use such values as the velocity of meshes covering such apertures.

20. The system according to claim 19, characterized in that it is provided in combination with an echographic apparatus having Doppler capabilities or a phase-contrast MRI apparatus for acquiring the values of the velocity of the blood at apertures crossing the boundary surface of the heart chamber to be transferred to the second input.

21. The system according to claim 17, characterized in being configured to be interfaced, or provided in combination, with an imaging apparatus for acquiring two-dimensional or three-dimensional images of a heart of a subject, the processing unit being configured to evaluate a geometry of an endocardial border.
